(19) United States
(12) Patent Application Publication
(10) Pub. No.: US 2010/0331412 A1
(43) Pub. Date: Dec. 30, 2010

(54) Two container skin care product

(76) Inventors: Patricia Lawler Kenet, New York, NY (US); Barney Kenet, New York, NY (US)

Correspondence Address: Davidson, Davidson & Kappel, LLC, 485 7th Avenue, 14th Floor, New York, NY, 10018, US

(21) Appl. No.: 12/661,950

(22) Filed: Mar. 26, 2010

Related U.S. Application Data
(60) Provisional application No. 61/211,406, filed on Mar. 30, 2009.

(51) Int. Cl. A61K 31/19; A61P 17/00; B65D 85/00; B65D 71/00

(52) U.S. Cl. 514/557; 206/459.1; 206/581

(57) ABSTRACT

A skin product for improving the skin includes a first container containing a first product, the first product including alpha hydroxy acid, and second container containing a second product, the second product including a combination of humectants, moisturizers and an AHA (glycolic acid) formulation.

This claims the benefit of U.S. Provisional Patent Application No. 61/211,406, filed Mar. 30, 2009 and hereby incorporated by reference herein.

### FIELD OF THE INVENTION

The present invention relates to dermatological products and to methods related to improving skin care.

### BACKGROUND OF THE INVENTION

The topical use of α-hydroxy acids and α-keto acids for treatment of various skin conditions is well known in the art. Such uses are described, for example, in U.S. Pat. Nos. 3,879,537; 4,105,783 and 4,363,815, all incorporated by reference herein.

U.S. Pat. No. 5,420,106 incroporated by reference herein discloses methods of enhancing skin permeation and skin retention of α-hydroxy acids for the treatment of dermatological disorders by applying a composition containing an amphoteric salt of an α-hydroxy acid, such as lactic, glycolic, citric, tartaric or malic acid and an amino acid, dipepdide, polypeptide or proteins, in a dermatologically acceptable carrier which contains 0.5 to 8% glycerol monocaprylate, 0 to 80% petrolatum and 0 to 30% isopropyl myristrate, the pH of the composition being from 3 to 9, and compositions for practicing such methods.

The article “Enhancing effect of alpha-hydroxy acids on ‘in vitro’ permeation across the human skin of compounds with different lipophilicity” by Copoví A, Díez-Sales 0, Herráez-Domínguez JV, Herráez-Domínguez M., Int J Pharm. 2006 May 11;314(1):31-6, incorporated by reference herein, describes the percutaneous penetration-enhancing effects of glycolic acid, lactic acid and sodium lauryl sulphate through the human epidermis was investigated using 5-fluorouracil as a hydrophilic model permeant and three compounds belonging to the phenylalcohols: 2-phenyl-ethanol, 4-phenyl-butanol and 5-phenyl-pentanol.

The article “A double blind controlled study of the vehicle on the skin susceptibility to stinging from lactic acid”, incorporated by reference herein, by Sahlin A., Edlund F., and Loden M., International Journal of Cosmetic Science, 2007 October;29(5):385-90, describes the effect of lactic acid on the skin.

The article “Effects of alpha-hydroxy acids on the human skin of Japanese subjects” by Yamamoto, Y., Uede K., Yonie N., Kishioka A., Ohtani T., and Furukawa F., J. Dermatol. 2006 January; 33(1): 16-22, incorporated by reference herein, describes the effect of alpha-hydroxy acids on the skin.

### SUMMARY OF THE INVENTION

The present invention provides a skin product for improving the skin, comprising:

a first container containing a first product, the first product including an alpha hydroxy acid, and

a second container containing a second product, the second product including a combination of humectants, moisturizers and an AHA (glycolic acid) formulation.

The skin product can include packaging holding both containers, and instructions directing the user to use the first and second products in a certain manner, for example, on alternating days.

The present invention also provides a method for using the skin product, comprising: directing a user to use the first product on certain days and the second product on other days. For example, the user can directed to use the first product Monday/Wednesday/Friday and the first container can be labeled with directions to use on those days. The user can be directed to use the second product on Tuesday, Thursday and Saturday for example, and the second container can be labeled with direction to sue on those days as well.

The first product advantageously can be buffered with ammonium hydroxide, and formulated in water in oil emulsion to reduce stinging.

The first product can decrease corneocyte adhesion, dissolve intercellular bridge, encourage desquamation and normalize keritinization.

The second product, preferably is a soap-free, syndet cleanser. The AHA formulation preferably is a very mild AHA formulation.

The first product, used for example on alternating days, enhances the effect of the second product and vice versa.

### BRIEF DESCRIPTION OF THE DRAWING

FIG. 1 shows a skin product according to the present invention.

### DETAILED DESCRIPTION

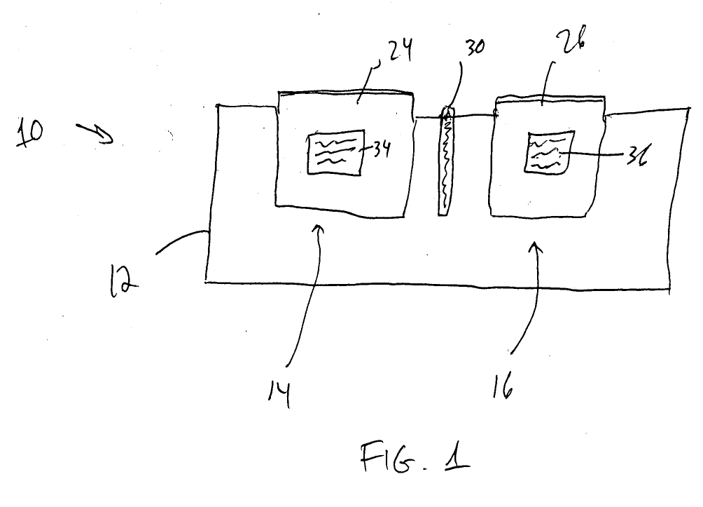

FIG. 1 shows a skin product 10, for example contained in a box 12 having a first receptacle 14 for a first container 24, and a second receptacle 16 for a second container 26.

First container 24 contains a first product, the first product including a combination of alpha hydroxy acid, lactic acid and salicylic acid.

Second container 26 contains a second product, the second product including a combination of humectants, moisturizers and an AHA (glycolic acid) formulation.

Instructions 30 can be provided separately from the container, and/or as labels 34, 36 on the containers 24, 26 respectively.

What is claimed is:

1. A skin product for improving the skin, comprising:
a first container containing a first product, the first product including an alpha hydroxy acid, and
a second container containing a second product, the second product including a combination of humectants, moisturizers and an AHA (glycolic acid) formulation.

2. The skin product as recited in claim 1 further comprising packaging holding both the first and second containers.

3. The skin product as recited in claim 1 further comprising instructions directing a user to use the first and second products in a certain manner.

4. A method for using the skin product as recited in claim 1, comprising: directing a user to use the first product on certain days and the second product on other days.

5. A skin product for improving the skin, comprising:
a first container containing a first product, the first product including a combination of alpha hydroxy acid, lactic acid and salicylic acid, and
a second container containing a second product, the second product including a combination of humectants, moisturizers and an AHA (glycolic acid) formulation.
